(19) United States
(12) Patent Application Publication
(10) Pub. No.: US 2017/0052250 A1
(43) Pub. Date: Feb. 23, 2017

(54) APPARATUS FOR REDUCING SIDE LOBES IN ULTRASONIC IMAGES USING NONLINEAR FILTER

(71) Applicants: WAYGENCE CO., LTD, Chuncheon-si Gangwon-do (KR); Daejin University Center for Educational Industrial Cooperation, Pocheon-si (KR)

(72) Inventors: Mok Kun JEONG, Seoul (KR); Sung Jae KWON, Seoul (KR)

(73) Assignees: WAYGENCE CO., LTD, Chuncheon-si Gangwon-do (KR); Daejin University Center for Educational Industrial Cooperation, Pocheon-si (KR)

(21) Appl. No.: 15/132,327

(22) Filed: Apr. 19, 2016

(30) Foreign Application Priority Data
Aug. 20, 2015 (KR) 10-2015-0117275

(51) Int. Cl. G01S 7/52; A61B 8/08; A61B 8/00

(52) U.S. Cl. CPC G01S 7/52047; A61B 8/4483; A61B 8/5269

(57) ABSTRACT

According to the present invention, ultrasonic image quality can be improved by removing side lobe signals using a nonlinear filter that subtracts the side lobe signals calculated from a summed signal resulting from the received channel signals obtained by focusing signals received via an array transducer having a plurality of receiving elements and uses the calculated magnitude of the side lobe signals as a filter coefficient. The present invention includes an array transducer to receive an ultrasonic signal reflected from an imaging point and to output the reflected ultrasonic signal as a channel signal of a corresponding receiving element, a focusing delay module to temporally align the channel signals of the receiving elements, a summation unit to sum the temporally aligned channel signals and output the summed signal in order to form an ultrasonic image, a side lobe computation module to calculate a waveform of a side lobe signal generated due to a leakage of the ultrasonic signal, and a filter unit to filter the summed signal of the summation unit based on the magnitude of the side lobe signal computed by the side lobe computation module in order to improve ultrasonic image quality.

### TECHNICAL FIELD

The present invention relates to an apparatus for reducing side lobes in ultrasonic images using a nonlinear filter, and more particularly, to an apparatus for reducing side lobes in ultrasonic images capable of improving ultrasonic image quality by removing a side lobe signal component using a nonlinear filter that estimates the side lobe signal using channel data resulting from applying a focusing delay to signals received by a plurality of receiving elements of an array transducer, subtracts the side lobe signal estimated from a receive focusing delayed channel data, and uses the magnitude of the estimated side lobe signal as the filter coefficients of a nonlinear side lobe reduction filter.

### BACKGROUND ART

Generally, ultrasonic images are used in diagnosing lesions. In medical ultrasonic imaging ultrasonic signals are transmitted via a transducer and the magnitude of ultrasonic signals received after being reflected from an inside of a human body is converted to brightness.

Despite the advantages of safety and real-time imaging capability, the ultrasonic images have a low resolution problem compared to other medical images. To solve this problem, a method of using an array transducer to focus ultrasonic waves of short pulse widths in order to transmit and receive the ultrasonic waves is being applied in a general medical ultrasonic imaging system.

Taking a close look at ultrasonic field in the ultrasonic focusing system, we can see that a main lobe is formed with respect to the scan line direction (i.e., axial direction) of ultrasonic image and that side lobes are formed at both sides of the main lobe due to leakage of ultrasonic signals. When the echoes from the target in the main lobe direction are received, signals from the target in the side lobe directions are also received with the result that the signals of the reflector in the side lobe act as noise in ultrasonic images and lower the resolution of the ultrasonic images.

Consequently, various attempts for reducing side lobes in ultrasonic images are recently being made, and details thereof are disclosed in detail in [Document 1], [Document 2], etc. as below.

However, in the cases of [Document 1] and [Document 2] below, since a method of applying weighting values to each received channel data is used, there is a problem of having to perform excess computation to reduce side lobes, and this problem is aggravated further as the number of channels increases.

### PRIOR ART DOCUMENT

### Patent Document

(Patent Document 1) [Document 1] Korean Unexamined Patent Application Publication No. 2009-0042152 (published on Apr. 29, 2009)

(Patent Document 2) [Document 2] Korean Registered Patent No. 971433 (announced on Jul. 14, 2010)

### Non-Patent Document

(Non-patent Document 1) None

### DISCLOSURE

### Technical Problem

The present invention has been devised to solve the above-mentioned problems of the prior art, and the present invention is directed to providing an apparatus for reducing adverse effects of side lobes in ultrasonic images using a nonlinear filter capable of filtering a side lobe signal component, in which a waveform of the side lobe signal is estimated after obtaining channel signals by focusing of signals received via an array transducer having a plurality of receiving elements, the estimated side lobe signal is subtracted in a process of summing the channel signals delayed for focusing, and the estimated side lobe signal is used as filter coefficients.

### Technical Solution

According to an embodiment of the present invention, an apparatus for reducing side lobes in ultrasonic images using a nonlinear filter includes an array transducer to receive an ultrasonic signal reflected from an imaging point from each of receiving elements to output the reflected ultrasonic signal as a channel signal of a corresponding receiving element, a focusing module to temporally align the channel signals of the receiving elements, a summation unit to add the temporally aligned channel signals and output the summed signal in order to form an ultrasonic image, a side lobe computation module to calculate a waveform of a side lobe signal generated due to a leakage of the ultrasonic signal, and a filter unit to filter the summed signal of the summation unit based on the magnitude of the side lobe signal computed by the side lobe computation module in order to improve ultrasonic image quality.

In addition, the filter unit may include a subtraction filter to perform a first filtering by subtracting the side lobe signal from the summed signal and a nonlinear filter to perform a second filtering whose input is fed from the signal firstly filtered by the subtraction filter.

In addition, the nonlinear filter may perform the second filtering using [Expression 1] below.

$${B}_{filtered} = ( \frac{1}{1 + γ ( \frac{{QF}_{p}}{{B}_{pixel}} )} ) \cdot ( {B}_{pixel} - {QF}_{p} ) \qquad [ Expression 1 ]$$

Here, Bₚᵢₓₑₗ is a brightness value of an ultrasonic image pixel, B _{filtered} is a brightness value of a filtered pixel, γ is a scale factor, and QFₚ is an image quality factor at each image pixel.

In addition, the image quality factor is the sum of the side lobe signal waveform calculated by the side lobe computation module in order to evaluate the ultrasonic image quality.

In addition, when calculating the spatial frequency of a sinusoidal signal waveform in the received channel data, the side lobe computation module uses zero-appending to extend the length of the received channel data.

### Advantageous Effects

As above, according to the present invention, an apparatus for reducing side lobes in ultrasonic images using a nonlinear filter is capable of improving an ultrasonic image quality by removing a side lobe component by filtering the side lobe component with a nonlinear filter that calculates a waveform of the side lobe signal in the received channel signals after applying focusing delay to the signals received via an array transducer having a plurality of receiving elements, subtracts the side lobe signal calculated using a subtraction filter in a process of summing up the channel signals delayed for focusing, and uses an output of the subtraction filter as an input to a second filter.

### DESCRIPTION OF DRAWINGS

FIG. 1 is a block diagram of an apparatus for reducing side lobes in ultrasonic images using a nonlinear filter according to the present invention.

FIG. 2A is a view for describing a relationship between an incident angle in an ultrasonic field and a spatial frequency of channel data.

FIG. 2B is a view for describing a spatial frequency.

FIG. 2C is a view illustrating a process of extending and computing a signal length in order to calculate a magnitude of a signal whose the spatial frequency is 1.5 cycles per aperture (CPA) where a first side lobe occurs.

FIGS. 3 to 13 are views illustrating test results for verifying a performance of a method of reducing side lobes in ultrasonic images using a nonlinear filter according to the present invention.

### MODES OF THE INVENTION

Hereinafter, an apparatus for reducing side lobes in ultrasonic images using a nonlinear filter according to an embodiment of the present invention will be described in detail with reference to the accompanying drawings.

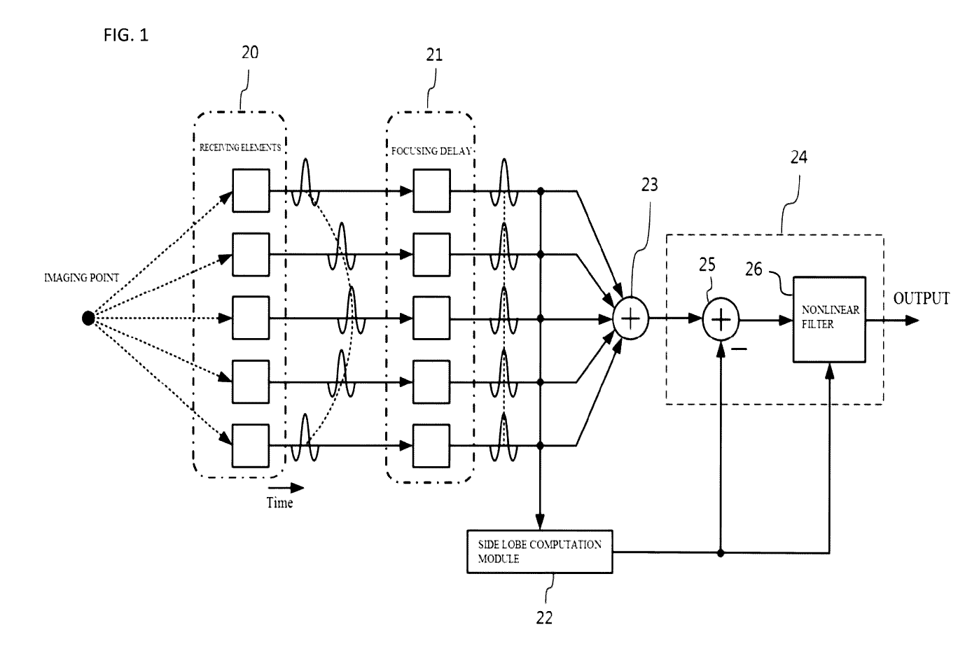

As illustrated in FIG. 1, an apparatus for reducing side lobes in ultrasonic images using a nonlinear filter according to the present invention includes an array transducer 20, a receive focusing delay module 21, a side lobe computation module 22, a summation unit 23, and a filter unit 24.

The array transducer 20 has a plurality of elements configured to transmit ultrasonic waves into a human body and receive signals reflected from a tissue of the human body.

The signals received from the human body tissue (i.e., an imaging point) arrive at each of the receiving elements at different times due to positions at which the receiving elements of the array transducer 20 are arranged. The focusing module 21 applies a time delay to each of the plurality of channel signals in which a difference in arrival time has occurred as above in order to perform focusing which temporally aligns the channel signals as if the channel signals had arrived at the same time.

In addition, the side lobe computation module 22 serves to compute a waveform of a side lobe signal using a spatial frequency characteristic of side lobe signal components included in the channel signals delayed for focusing by a method to be described below.

The filter unit 24 may improve ultrasonic image quality using a plurality of filters 25 and 26 that remove a side lobe signal component using the calculated waveform of the side lobe signal. A subtraction filter 25 performs a first filtering by subtracting the sum of the waveform of side lobe signal calculated by the side lobe computation module 22 from a summed signal of the summation unit 23, and a nonlinear filter 26 performs a second filtering on the signal firstly filtered by the subtraction filter 25.

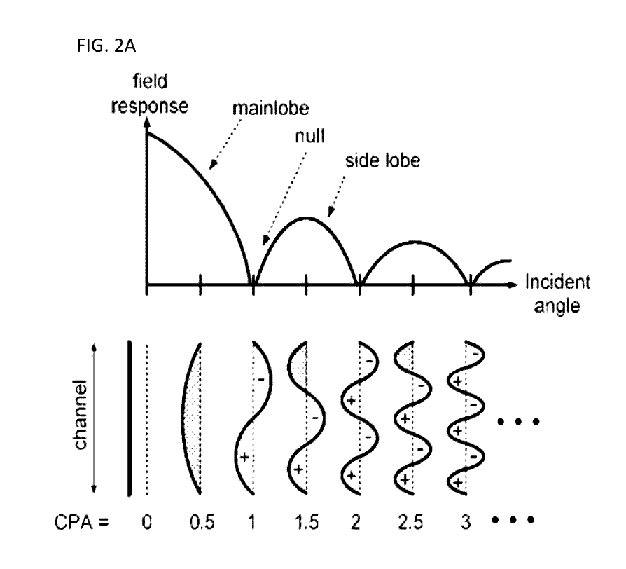

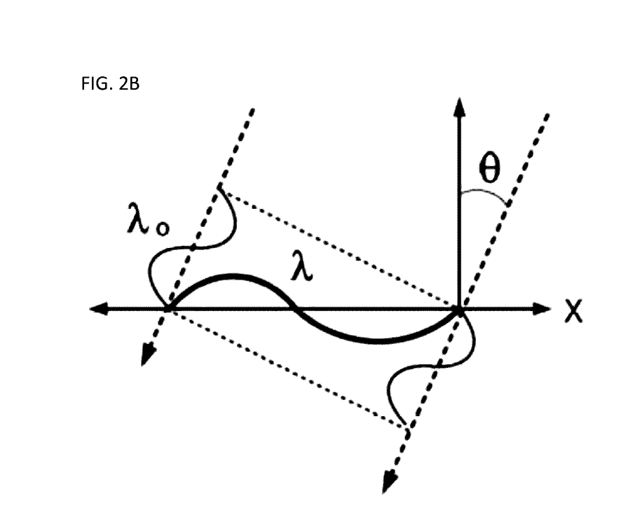

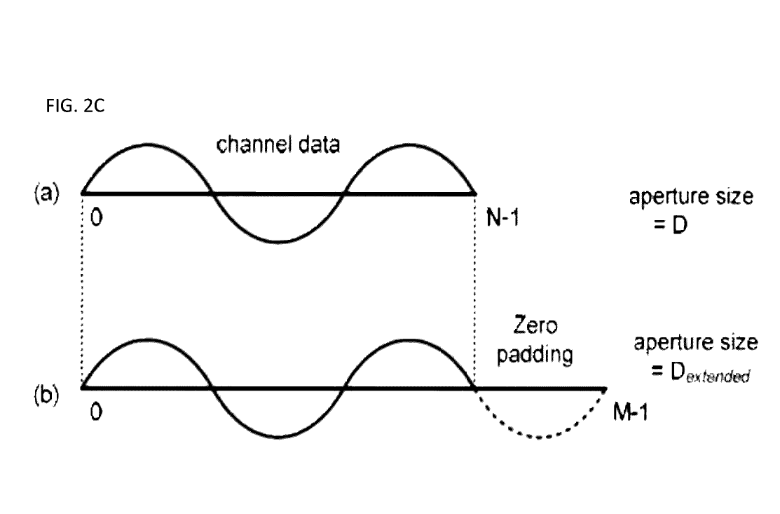

As illustrated in FIG. 2A, an ultrasound field characteristic obtained from an imaging region of a general ultrasonic focusing system is that a main lobe is formed with respect to a scan line direction of a transducer and side lobes are formed due to a leakage of ultrasonic signals.

In this manner, when the reflected signals impinge on the transducer from a direction adjacent to and at random angles with the scan line direction of the transducer, the signals are incident on the receiving elements with different phases.

Consequently, the signals incident from the random incident angles are shown as signals having a specific frequency referred to as a spatial frequency when viewed from the transducer.

The spatial frequency will be described with reference to FIG. 2B. When a continuous wave (CW) ultrasound field is incident on the receiving elements of the transducer at a long distance while having a predetermined incident angle θ, the CW sound field arrives at the array elements with different phases depending on the receiving positions and takes a sinusoidal form across the receiving transducer aperture.

A wavelength λ of a sinusoid observed at a position in the x-axis when an ultrasonic wave having a wavelength λ₀ is incident at a predetermined angle θ can be expressed as [Equation 1].

$$λ = \frac{{λ}_{o}}{sin θ} \qquad [ Equation 1 ]$$

The number of cycles of a sinusoid appearing in a channel signal received from a receiving element of a transducer whose size is D is defined as a cycle per aperture (CPA), and this can be written as [Equation 2] below. Here, the CPA refers to a spatial frequency of a signal periodically appearing across the aperture of a transducer array.

$$C P A = \frac{D}{λ} = \frac{D}{{λ}_{o}} sin θ \qquad [ Equation 2 ]$$

According to [Equation 2], the spatial frequency of the sinusoid appearing on the receiving element of the transducer whose size is D varies in accordance with the incident angle θ.

The received channel signal can be written as [Equation 3] below.

$$s ( n ) = {\sum }_{k = 0}^{N - 1} {x}_{k} ( n )$$

Here, Xₖ is a channel signal received by a k^{th} receiving element at time n, and the sum of all of the channel signals is s(n). Signals simultaneously incident on a receive channel from various directions may be modeled as a sum of sinusoids having various spatial frequencies in accordance with an incident angle.

The discrete Fourier transform of [Equation 3] can be shown as [Equation 4] below.

$$\begin{matrix}{X}_{m} ( n ) = {\sum }_{k = 0}^{N - 1} {x}_{k} ( n ) \cdot {e}^{- j \frac{2 π k m }{N}} & [ Equation 4 ] \\ {x}_{k} ( n ) = \frac{1}{N} {\sum }_{m = 0}^{N - 1} {X}_{m} ( n ) \cdot {e}^{j \frac{2 π k m }{N}} & \end{matrix}$$

[Equation 4] is modeled by summing sinusoids having frequencies which are integer multiples of the reciprocal of the channel length, and the signals summed in a receive focusing process can be expressed as [Equation 5] below using [Equation 3] and [Equation 4].

$$\begin{matrix}s ( n ) = {\sum }_{k = 0}^{N - 1} {x}_{k} ( n ) = {\sum }_{k = 0}^{N - 1} \{ \frac{1}{N} {\sum }_{m = 0}^{N - 1} {X}_{m} ( n ) \cdot {e}^{j \frac{2 π k m }{N}} \} \\ = {\sum }_{m = 0}^{N - 1} {X}_{m} ( n ) \cdot \{ \frac{1}{N} {\sum }_{k = 0}^{N - 1} {e}^{j \frac{2 π k m }{N}} \} \\ = {X}_{o} ( n )\end{matrix} \qquad [ Equation 5 ]$$

Consequently, sinusoidal components having frequencies which are integer multiples of the reciprocal of the channel length are removed from a finally focused signal and only a direct current (DC) component remains. The DC component of the channel signal becomes a signal that arrives at all receive channels with the same phase.

Referring to FIG. 2A, a horizontal axis represents an incident angle, and the incident angle is related to the spatial frequency by [Equation 2]. Since a channel signal that has an integer CPA is removed in the focusing process, a null of a sound field appears when the incident angle is at a specific position (a position at which the CPA becomes an integer) in an ultrasound field characteristic. In contrast, when the channel signals are summed at an incident angle of frequency for which the CPA is (integer+0.5), the peak of the summed signal appears at the incident angle of side lobes since the shaded area (half-wavelength components) in the channel data of FIG. 2A portions remain. Consequently, when signal components whose spatial frequency is (integer+0.5) CPA are calculated from the channel signals, the waveform of the side lobes may be approximately estimated.

Depending on the incident angle, the received channel signals may be modeled as a sum of sinusoids having various frequencies. Modeling the channel signals as a sum of sinusoids Eₘ(n) having integer frequencies and sinusoids Oₘ(n) having frequencies of (integer+0.5), we obtain [Equation 6] as follows.

$$s ( n ) = {\sum }_{k = 0}^{N - 1} \{ {\sum }_{m = 1}^{N - 1} {E}_{m} ( n ) \cdot {e}^{j \frac{2 π m k }{N}} + {\sum }_{m = 1}^{N - 1} {O}_{m} ( n ) \cdot {e}^{j \frac{2 π ( m + 0.5 ) k }{N}} \} + {E}_{o} ( n ) \qquad [ Equation 6 ]$$

E_{O}(n) represents a DC component of a channel, k represents a channel number, m represents a null or a side lobe index, and n represents a sampling time.

As shown in [Equation 5], when the sinusoids Eₘ(n) having the integer frequencies are summed, the sum becomes zero such that they are removed in a focusing process. However, when the sinusoids Oₘ(n) having the frequencies of (integer+0.5) are summed in the focusing process, the positive and negative signals cannot be canceled out and remain as a side lobe.

A method for calculating a signal component having a frequency of (integer+0.5) from channel signals will be described.

The conventional discrete Fourier transform is not appropriate for accurately estimating a waveform of a side lobe signal since it calculates a sinusoidal signal having an integer spatial frequency. Consequently, the data length needs to be extended by appending an appropriate number of zeros in order to calculate a magnitude of a sinusoid having a frequency of (integer+0.5) using the orthogonality of sinusoids. This process is referred to as zero appending.

FIG. 2C illustrates an example of extending the data length in order to calculate a signal waveform whose spatial frequency is 1.5 CPA at which a first side lobe appears. For the signal that has 1.5 CPA to become a signal with 2 CPA, zeros are appended at the end of the channel data to extend the length of the channel data. Since the extended sinusoid becomes a signal having an integer frequency in the extended channel length, the magnitude of the sinusoidal signal can be calculated using the discrete Fourier transform. Since the calculated result has a complex amplitude, a channel data waveform of a side lobe can be estimated by taking the inverse discrete Fourier transform.

For example, in the case of a system having 64 channels (when there are 64 receiving elements of the transducer) an extended data length of the first side lobe is given by [Equation 7] below.

$$round ( 64 \times \frac{2}{1.5} ) = 85 \qquad [ Equation 7 ]$$

Here, round( ) is a function that makes the argument take an integer value by the process of rounding off. Since CPA=2 in the extended data when an amplitude of a first side lobe is calculated using the orthogonality principle, this can be written as [Equation 8] below.

$${A}_{1} ( n ) = {\sum }_{k = 0}^{M - 1} {x}_{k} ( n ) \cdot {e}^{- j \frac{4 π k }{M}} , \\ where {x}_{k} ( n ) = 0 , k = N , … , M - 1 \qquad [ Equation 8 ]$$

Here, M=85. Aᵢ(n) corresponds to a complex amplitude of a first side lobe. From [Equation 8] the channel data waveform of the first side lobe can be expressed as [Equation 9] below when CPA=1.5 is applied to the channel data in which the length N=64.

$${s}_{1} ( n , k ) = {A}_{1} ( n ) \cdot {e}^{j \frac{3 π k }{N}} , k = 0 , … , N - 1 \qquad [ Equation 9 ]$$

A waveform of a channel signal may also be calculated for other sinusoids in channel data having the frequency of (integer+0.5) using the same method after appending zeroes whose number corresponds to a half-wavelength of each spatial frequency. Since all side lobe signals have different frequency components, each of them should be separately calculated after appropriately extending to a different channel data length. Consequently, a case of calculating all the waveforms of side lobes up to a degree P and adding all of the channel data can be represented as follows:

$${sidelobe}_{p} ( n ) = {\sum }_{m = 1}^{p} ( {\sum }_{k = 0}^{N - 1} {s}_{m} ( n , k ) ) = {\sum }_{m = 1}^{p} ( {\sum }_{k = 0}^{N - 1} {A}_{m} ( n ) \cdot {e}^{j \frac{2 πk ( m + 0.5 ) }{N}} ) \qquad [ Equation 10 ]$$

sidelobeₚ(n) is a sum of side lobe signals up to the degree P at a corresponding pixel, where the index P indicates the Pth side lobe, and this value is a clutter component that degrades ultrasonic image quality. Consequently, the ultrasonic image quality may be improved by removing the clutter component.

The side lobe computation module 22 may compute the magnitude of a side lobe signal by following the above computation process.

The sum of side lobe signals in the received channel signal calculated using [Equation 10] corresponds to a magnitude of a signal that degrades image quality. Thus, to evaluate the image quality, an image quality factor may be defined as QF, which can be expressed as follows:

QFₚ=sidelobeₚ [Equation 11]

In [Equation 11], a time index n in the depth direction is removed for generalization.

The sum of side lobe signals calculated by the side lobe computation module 22 may be subtracted from the channel data in the focusing process to reduce the adverse effects of the side lobes. This operation can be expressed as:

B_{filtered}=Bₚᵢₓₑₗ−QFₚ [Equation 12]

The subtraction filter 25 of the filter unit 24 may perform a first filtering, which subtracts the sum of the side lobe signals calculated by applying [Equation 12].

Considering the fact that a wideband transmit pulse is usually used in an ultrasonic imaging system, the ultrasonic image quality cannot be sufficiently improved when we employ the subtraction process only.

To cope with the above problem, the filter unit 24 applies the nonlinear filter 26. That is, the nonlinear filter 26 may be applied as a filtering means capable of further increasing a filtering effect of removing the side lobe components from the ultrasonic images.

Hereinafter, a method of designing the nonlinear filter 26 will be described.

When it is expected that a poor image quality may result due to an increase of clutter in ultrasonic images, a nonlinear filter can be designed so as to decrease the brightness of a corresponding pixel using the following equation:

$${B}_{filtered} = ( \frac{{B}_{pixel}}{{B}_{pixel} + γ {QF}_{p}} ) \cdot {B}_{pixel} = ( \frac{1}{1 + γ ( \frac{{QF}_{p}}{{B}_{pixel}} )} ) \cdot {B}_{pixel} \qquad [ Equation 13 ]$$

Here, Bₚᵢₓₑₗ is a brightness value of an ultrasonic image pixel, and B_{filtered} is a brightness value of a filtered pixel. The effect of the filter may be controlled using the scale factor γ and the image quality factor QF_{P} of the degree P.

The nonlinear filter designed as in [Equation 13] suppress the magnitude of a signal coming from a direction of a side lobe at each image pixel.

The clutter may be further reduced when the subtraction filter 25 to which [Equation 12] is applied and the nonlinear filter 26 to which [Equation 13] is applied are combined. That is, when a nonlinear filter in which an output of [Equation 12] is input to [Equation 13] is designed, this can be represented as follows:

$${B}_{filtered} = ( \frac{1}{1 + γ ( \frac{{QF}_{p}}{{B}_{pixel}} )} ) \cdot ( {B}_{pixel} - {QF}_{p} ) \qquad [ Equation 14 ]$$

The nonlinear filter 26 whose input is the output of the subtraction filter 25 is applied according to [Equation 14]. The ultrasonic image quality may be further improved by more faithfully removing the side lobe components.

Hereinafter, a test result using computer simulations to verify an effect of removing side lobes from ultrasonic images according to the present invention will be described.

A wideband ultrasonic pulse was used, and a transmit pulse had a Gaussian shape with a duration of 5 cycles. Conditions for performing the computer simulation are shown in [Table 1] below.

TABLE 1

| Transducer | Linear array | |
|---|---|---|
| Center frequency | 7.5 | MHz |
| Element pitch | 0.3048 | mm |
| Number of receive channels | 64 | elements |
| Transmit focal depth | 25 | mm |

### Example 1

A point spread function (PSF) of a wire target at a 35 mm depth in a phantom used for observing an effect of filtering side lobe signals was calculated. Here, an image had a 10-mm width and a 2-mm height, the image was log compressed over a dynamic range of 50 dB, and apodization was not applied to the image.

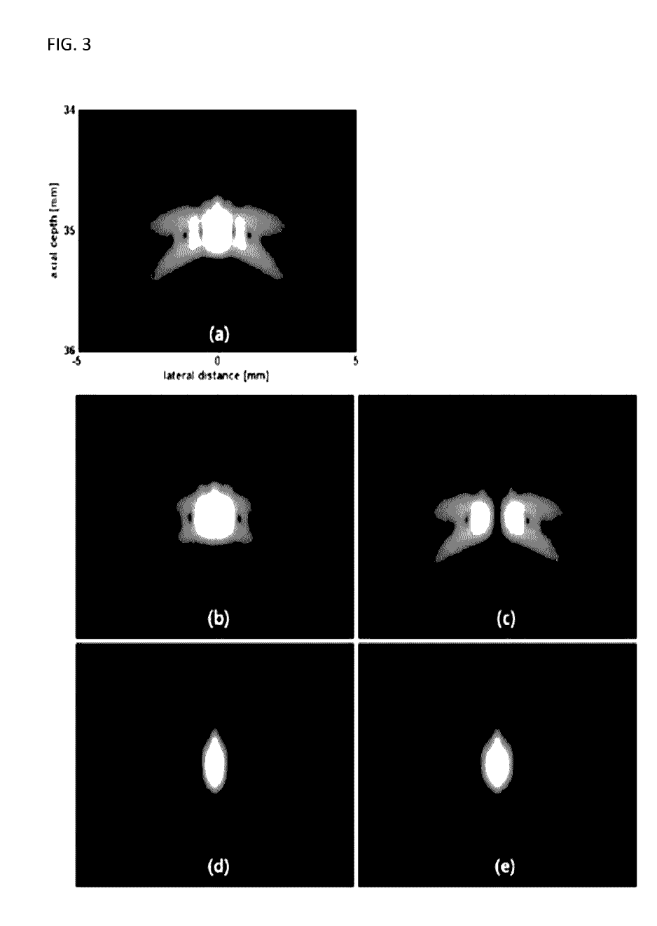

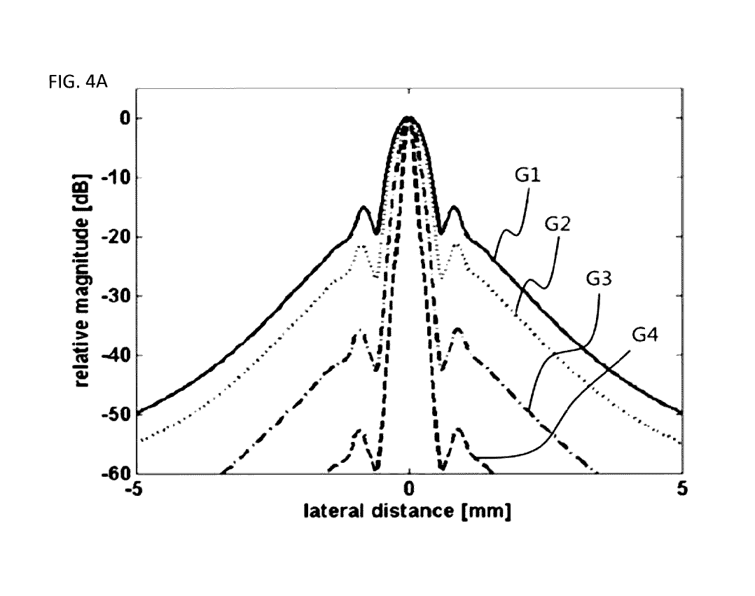

FIG. 3(a) is the PSF according to the prior art. FIG. 3(c) is an image QF₂₀ in which side lobes up to a twentieth side lobe are calculated and all of them are summed. FIG. 3(b) is an image from which side lobe components are subtracted using the subtraction filter 25 to which [Equation 12] is applied. Here, although the side lobe components were decreased, it was shown that the width of the main lobe was increased. FIG. 3(d) is a case where the nonlinear filter designed according to [Equation 13] is applied and FIG. 3(e) is a case where the nonlinear filter designed according to [Equation 14] is applied. In FIG. 3(d) and FIG. 3(e), the scale factor γ was set to 10.

### Example 2

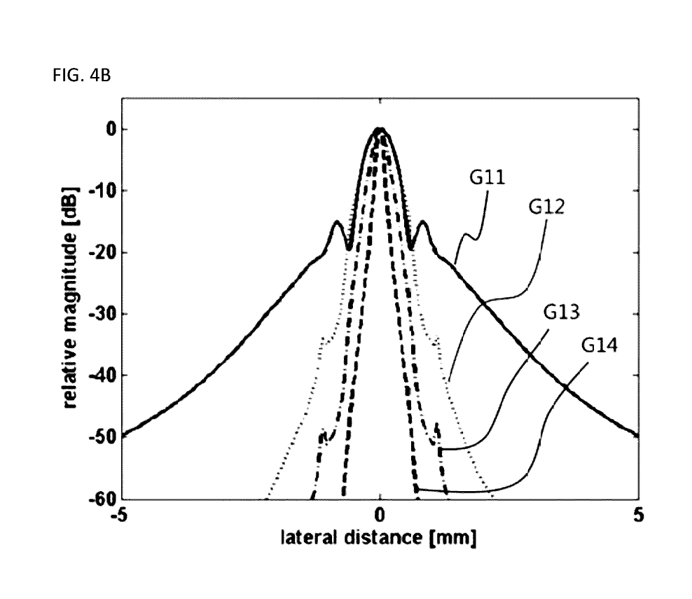

FIGS. 4A and 4B show the result of comparing the lateral ultrasound field characteristics of a wire target in order to examine the effects of scale factors γ using [Equation 13] and [Equation 14], respectively.

FIG. 4A compares cases in which the prior art and the nonlinear filter using [Equation 13] were respectively applied, and FIG. 4B compares cases in which the prior art and the nonlinear filter using [Equation 14] were respectively applied.

FIG. 4A shows the ultrasound field characteristics, where the solid line G1 is obtained using the prior art, and the dotted G2, dash-dot G3, and dashed G4 lines represent the case of applying the nonlinear filter of [Equation 13] in which the scale factor is set to 1, 10, and 100, respectively.

FIG. 4B shows the ultrasound field characteristics, where the solid line G11 is obtained using the prior art, and the dotted G12, dash-dot G13, and dashed G14 lines represent the case of applying the nonlinear filter of [Equation 14] in which the scale factor is set to 1, 10, and 100, respectively.

As shown in FIGS. 4A and 4B, an effect of reducing side lobes increased as the scale factor γ was increased, and the effect of reducing side lobes was better when the nonlinear filter of [Equation 14] was applied than when the nonlinear filter of [Equation 13] was applied.

### Example 3

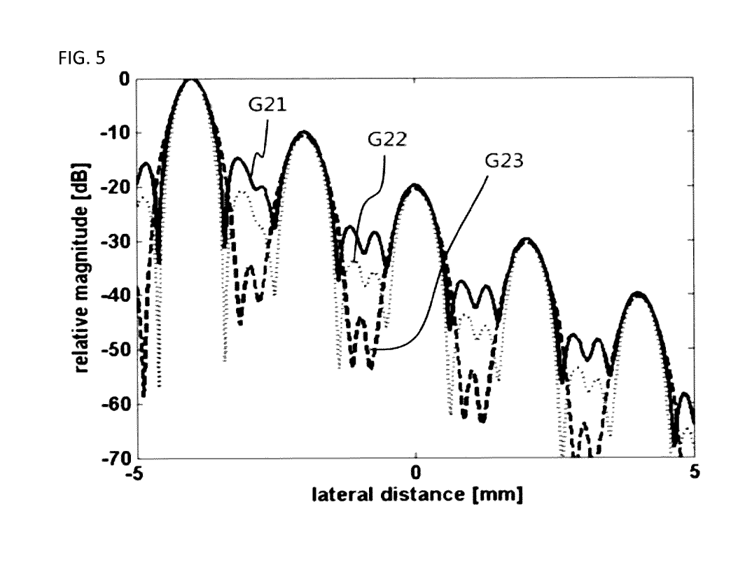

FIG. 5 shows a comparison of the lateral ultrasound field characteristics by arranging wire targets with 10-dB differences in reflectivity at 2-mm intervals inside a phantom. In graphs G21, G22, and G23, a scale factor γ=1 was used under conditions of similarly maintaining the width of the main lobe and maintaining the linearity of the filter output. The prior art is shown in the solid line G21, an application of the nonlinear filter of [Equation 13] is shown in the dotted line G22, and an application of the nonlinear filter of [Equation 14] is shown in the dashed line G23. Although the shapes of side lobe signals are shown to be similar due to the same intervals between all the wire targets, the effect of reducing side lobes was better when the nonlinear filter of [Equation 14] was applied than when the nonlinear filter of [Equation 13] was applied.

### Example 4

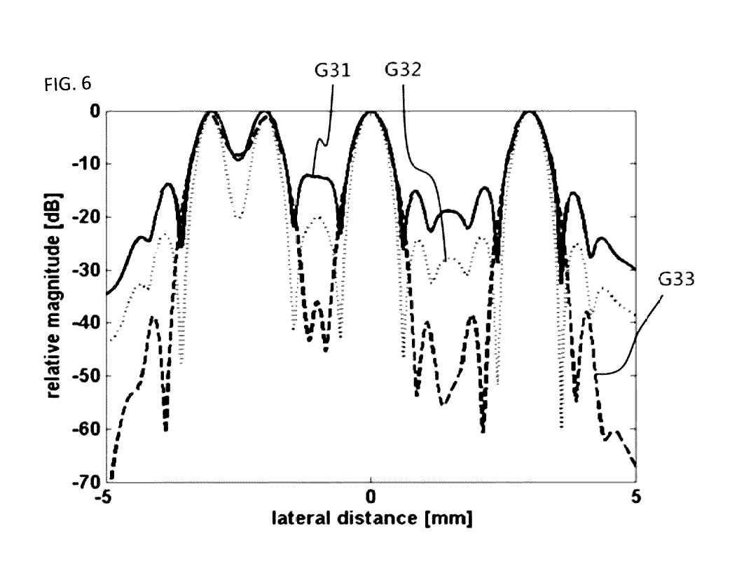

FIG. 6 shows a comparison of the lateral ultrasound field characteristics by placing wire targets at intervals of 1, 2, and 3 mm. A scale factor γ=2 was used under a condition of similarly maintaining the main lobe shape. Although the side lobe shapes are shown to be different depending on intervals between point targets, the effect of reducing side lobes was better when the nonlinear filter of [Equation 14] was applied as in FIG. 5 of (Example 3). Here, the prior art is shown in the solid line G31, an application of the nonlinear filter of [Equation 13] is shown in the dotted line G32, and an application of the nonlinear filter of [Equation 14] is shown in the dashed line G33.

### Example 5

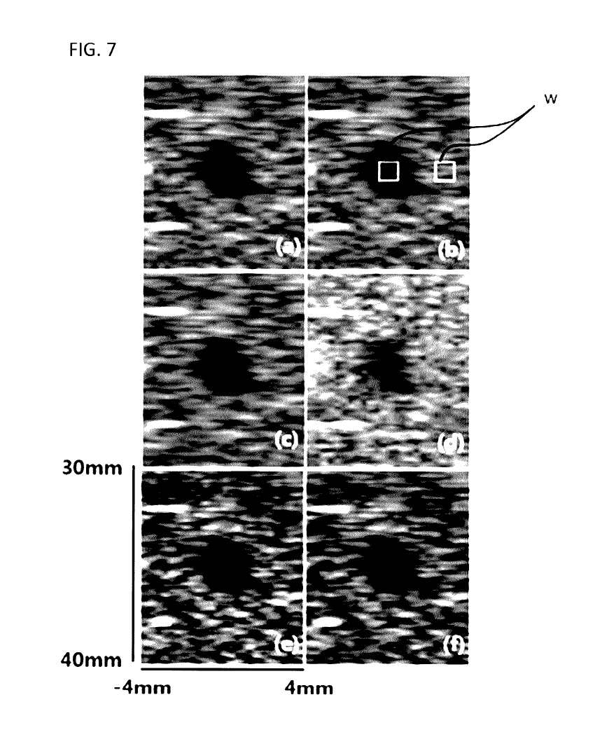

In FIG. 7, images of a 3-mm diameter anechoic cyst in random scatterers were obtained. The cyst was placed at a 35-mm depth and the wire targets were placed at depths of 32 and 38 mm.

FIG. 7(a) is an image according to the prior art, and the width of the cyst region is shown to be decreased due to the lateral ultrasound field characteristic. In FIG. 7(b), the white windows represent a background region and a cyst region, which are used in calculating a signal-to-noise ratio (SNR) in the prior art.

FIG. 7(c) is an image from which side lobe components are subtracted and is shown to be slightly darker due to a decrease in side lobes inside the cyst.

FIG. 7(d) is a quality factor image.

FIG. 7(e) is an image obtained using the nonlinear filter of [Equation 13] with a scale factor γ=10. FIG. 7(f) shows a case of using the nonlinear filter of [Equation 14] with a scale factor γ=10. Although the cyst region is shown to be darker, the speckle region is shown to be granular.

The results of calculating contrast, contrast-to-noise ratio (CNR), and SNR in the regions marked with white windows in FIGS. 7(b), 7(c), 7(e), and 7(f) are shown in [Table 2] below.

TABLE 2

| | Contrast | CNR | SNR |
|---|---|---|---|
| FIG. 7(b) | −12.9 | 4.01 | 1.65 |
| FIG. 7(c) | −25.4 | 5.51 | 0.60 |
| FIG. 7(e) | −29.7 | 3.42 | 0.47 |
| FIG. 7(f) | −60.4 | 3.59 | 0.11 |

Although the contrast significantly increases when the side lobes were removed as shown in FIG. 7(c), the speckle pattern is granular so that the CNR and SNR decrease. The other results also represent the processed cases to decrease the clutter, where it can be seen that the images are granular.

### Example 6

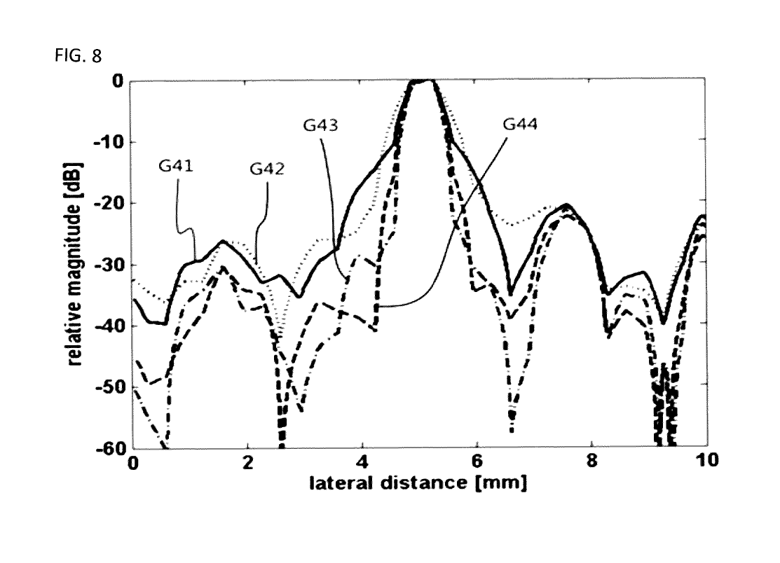

A wire target placed at a 38-mm depth in a background of random scatterers was imaged, and FIG. 8 illustrates the lateral ultrasound field characteristic after filtering.

The prior art is shown in the solid line G41, a case of subtracting side lobe components is shown in the dotted line G42, an application of the nonlinear filter of [Equation 13] is shown in the dash-dot line G43, and an application of the nonlinear filter of [Equation 14] is shown in the dashed line G44, where a scale factor γ=10 was used in each of the last three cases. Although the width of the main lobe increased as shown in the dotted line G42 when the side lobe signals were subtracted, it can be seen that both the main lobe width and the side lobes were decreased as can be seen in the dash-dot line G43 and the dashed line G44, which correspond to [Equation 13] and [Equation 14], respectively.

### Example 7

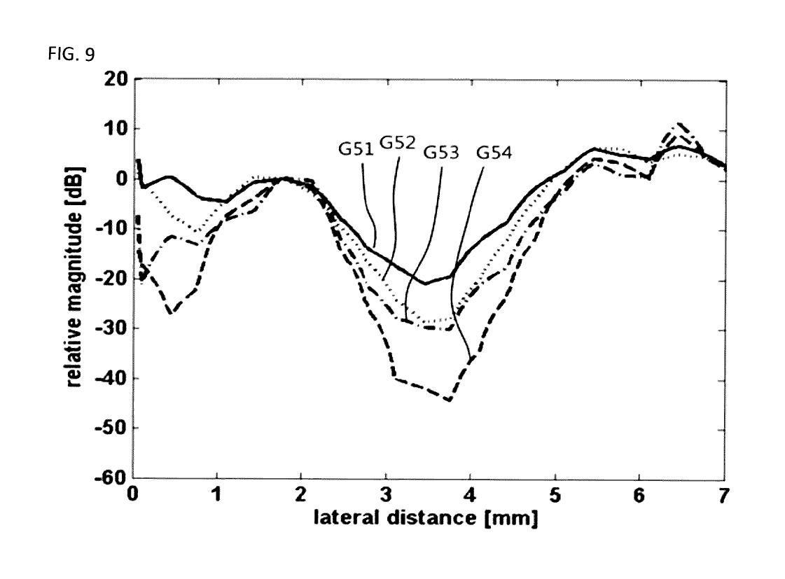

FIG. 9 illustrates a case of comparing the reflectivity of an anechoic region inside a cyst. In all of the graphs G52, G53, and G54 from which side lobe components were filtered, it was shown that the clutter was faithfully removed.

The prior art is shown in the solid line G51, a case of subtracting the side lobe components is shown in the dotted line G52, an application of the nonlinear filter of [Equation 13] is shown in the dash-dot line G53, and an application of the nonlinear filter of [Equation 14] is shown in the dashed line G54. A scale factor γ=10 was used in each of the last three cases.

### Example 8

Test conditions for obtaining channel data from wire targets in a water tank and a human body are presented in [Table 3] below.

TABLE 3

| Transducer | Linear array | |
|---|---|---|
| Center frequency | 7.5 | MHz |
| Element pitch | 0.3048 | mm |
| Number of receive channels | 64 | elements |
| Transmit focal depth | 50 | mm |

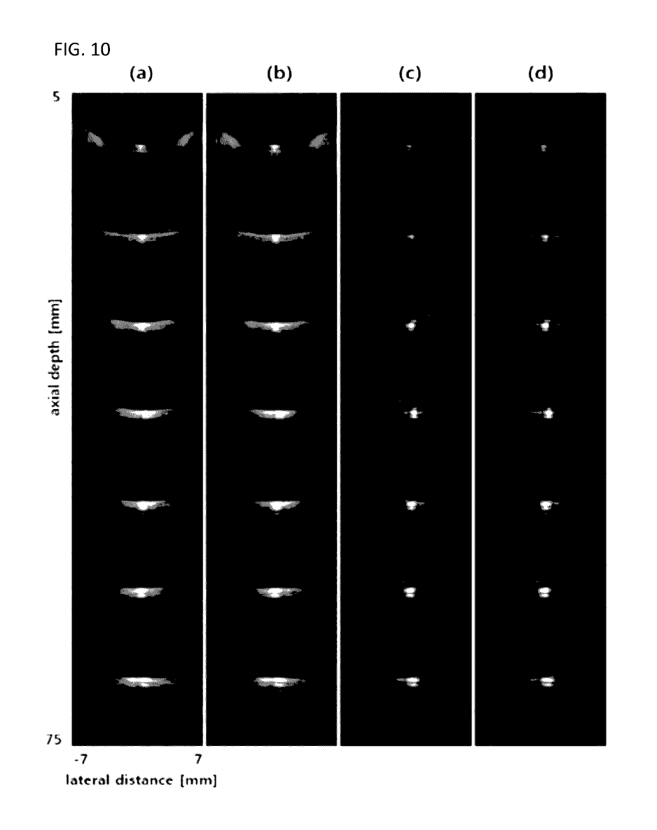

The wire targets in the water tank were vertically placed at intervals of 10 mm over a depth of 10 mm to 70 mm. FIG. 10(a) illustrates a case of the prior art, FIG. 10(b) illustrates a case in which side lobe components were subtracted, FIG. 10(c) illustrates a case of applying the nonlinear filter of [Equation 13], and FIG. 10(d) illustrates a case of applying the nonlinear filter of [Equation 14]. A scale factor γ=10 was used in FIGS. 10(c) and 10(d).

In FIG. 10(b), it can be seen that X-shaped side lobes have been removed around the wire targets at depths of 10 mm and 20 mm. The regions over which side lobe components are present were reduced in FIGS. 10(c) and 10(d).

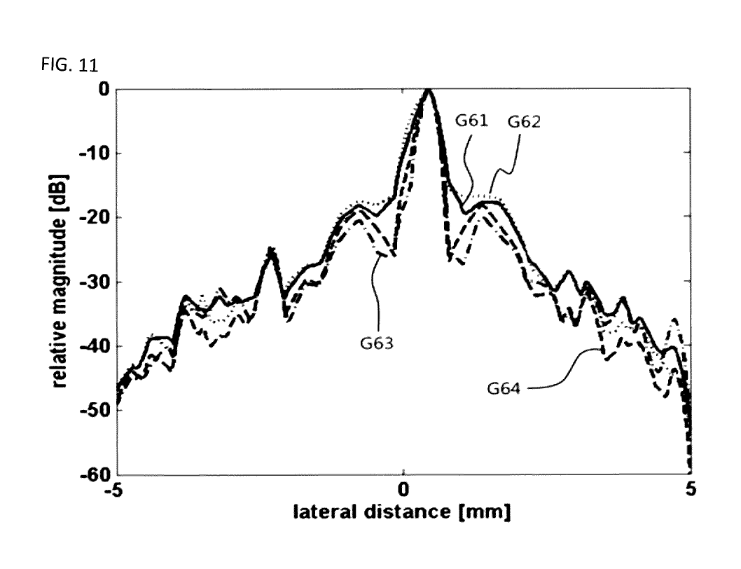

FIG. 11 compares the maximum values of side lobe components of the ultrasound field in the lateral direction for a wire target at a depth of 20 mm, where (a) is the PSF in accordance with the prior art, which is shown in the solid line G61; (b) is a case of subtracting the side lobe components, which is shown in the dotted line G62; (c) is a case of applying the nonlinear filter of [Equation 13], which is shown in the dash-dot line G63; and (d) is a case of applying the nonlinear filter of [Equation 14], which is shown in the dashed line G64. A scale factor γ=10 was used in FIGS. 11(c) and 11(d).

### Example 9

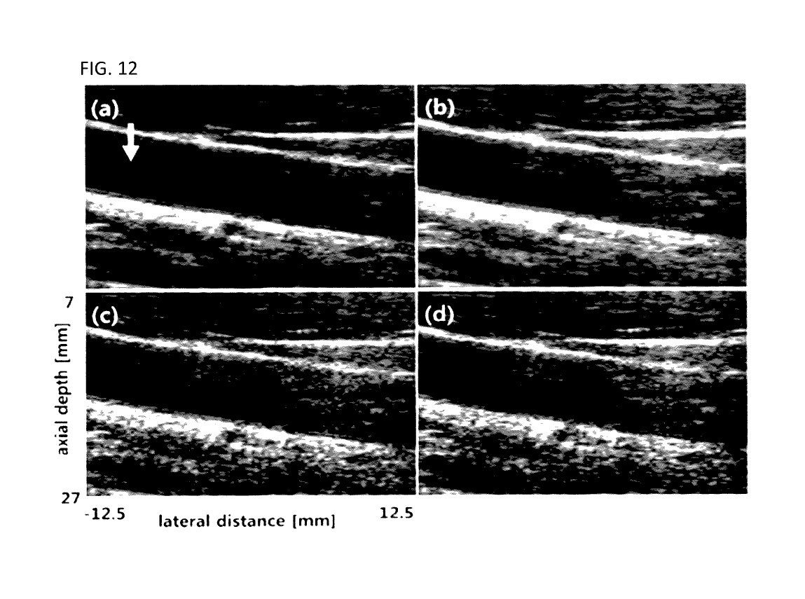

FIG. 12 shows the vascular images of a neck portion of a human body. A log compression of 60 dB was performed, and FIG. 12(a) illustrates a case of the prior art, FIG. 12(b) illustrates a case in which side lobe components were subtracted, FIG. 12(c) illustrates a case of applying the nonlinear filter of [Equation 13], and FIG. 12(d) illustrates a case of applying the nonlinear filter of [Equation 14]. A scale factor γ=10 was used in FIGS. 12(c) and 12(d).

The area indicated by an arrow in FIG. 12(a) represents the interior of a blood vessel. Since the reflectivity inside the blood vessel is low, noise appearing here may be deemed as being caused by the clutter. It can be seen that the magnitude of the clutter has been reduced in each of the filtering cases shown in FIGS. 12(b), 12(c), and 12(d).

### Example 10

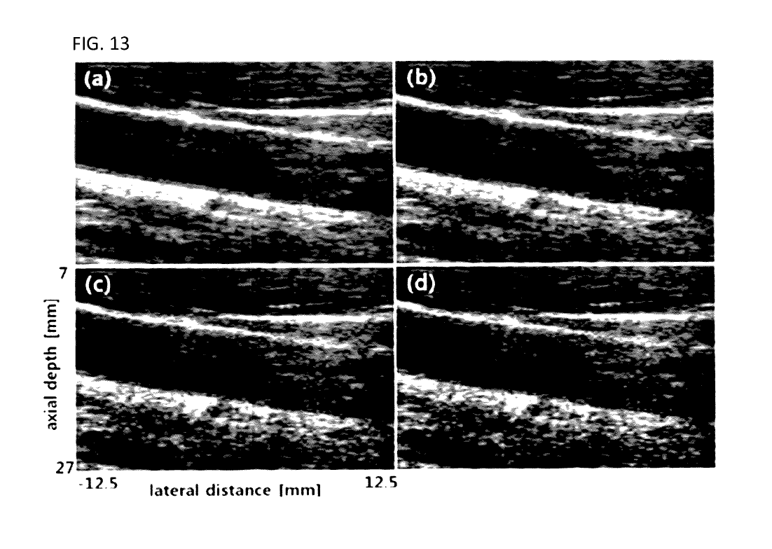

FIG. 13 shows the vascular images of a neck portion of a human body to which the nonlinear filter of [Equation 14] was applied. A log compression of 60 dB was performed and different values of the scale factor γ were used.

A scale factor γ=0.01, 0.1, 1, and 10 was used in obtaining FIG. 13(a), 13(b), 13(c), 13(d), respectively.

The granular pattern of the images gets finer as the scale factor γ is increased. Consequently, when making an actual diagnosis, it is preferable that an ultrasonographer control the ultrasonic image quality by adjusting the scale factor γ while observing the image quality.

Although particular embodiments of the present invention have been illustrated and described above, it should be apparent to those of ordinary skill in the art that the technical spirit of the present invention is not limited to the accompanying drawings and the above description and that various modifications are possible within the scope not departing from the spirit of the present invention. Also, the modifications should be viewed as belonging to the claims of the present invention within the scope not contrary to the spirit of the present invention.

1. An apparatus for reducing side lobes in ultrasonic images using a nonlinear filter, the apparatus comprising:
an array transducer configured to receive an ultrasonic signal reflected from an imaging point and to output the reflected ultrasonic signal as a channel signal of a corresponding receiving element;
a focusing delay module configured to temporally align the channel signals of the receiving elements;
a summation unit configured to add the temporally aligned channel signals and to output the summed signal in order to form an ultrasonic image;
a side lobe computation module configured to calculate a waveform of a side lobe signal generated due to a leakage of the ultrasonic signal; and
a filter unit configured to filter the summed signal of the summation unit based on the magnitude of the side lobe signal computed by the side lobe computation module in order to improve ultrasonic image quality.

2. The apparatus according to claim 1, wherein the filter unit comprises a subtraction filter configured to perform a first filtering by subtracting the calculated side lobe signal from the summed signal and a nonlinear filter configured to perform a second filtering on the signal firstly filtered by the subtraction filter.

3. The apparatus according to claim 2, wherein the nonlinear filter performs the second filtering using [Expression 1] below:
$${B}_{filtered} = ( \frac{1}{1 + γ ( \frac{{QF}_{p}}{{B}_{pixel}} )} ) \cdot ( {B}_{pixel} - {QF}_{p} ) \qquad [ Expression 1 ]$$
where Bₚᵢₓₑₗ a brightness value of an ultrasonic image pixel, B_{filtered} is a brightness value of a filtered pixel, γ is a scale factor, and QFₚ is an image quality factor at each pixel.

4. The apparatus according to claim 3, wherein the image quality factor is the sum of the side lobe signal waveform calculated by the side lobe computation module in order to evaluate the ultrasonic image quality.

5. The apparatus according to claim 1, wherein, when calculating the spatial frequency of a sinusoidal signal waveform in the received channel data, the side lobe computation module uses zero-appending to extend the length of the received channel data.
